(19) United States
(12) Patent Application Publication
(10) Pub. No.: US 2007/0093435 A1
(43) Pub. Date: Apr. 26, 2007

(54) Modulation of cell phenotype by inhibitory rna

(76) Inventors: Peter Andrews, Sheffield (GB); Paul Gokhale, Sheffield (GB); Jim Walsh, Sheffield (GB); Maryam Matin, (US)

Correspondence Address: KLARQUIST SPARKMAN, LLP, 121 SW SALMON STREET, SUITE 1600, PORTLAND, OR, 97204, US

(21) Appl. No.: 10/550,499

(22) PCT Filed: Mar. 25, 2004
(86) PCT No.: PCT/GB04/01374

§ 371 Date: Jan. 10, 2007

(30) Foreign Application Priority Data
Mar. 28, 2003 (GB) 0307206.3

(51) Int. Cl. A61K 48/00; C07H 21/02; C12N 15/00

(52) U.S. Cl. 514/44; 536/23.1; 435/320.1

(57) ABSTRACT

We describe a method to inhibit cell division of hyperproliferative cells, typically cancer cells, by introducing inhibitory RNA (RNAi) into said cells to ablate mRNAs that encode polypeptides involved in cellular processes and including RNAi molecules and vectors including transcription cassettes encoding said RNAi molecules. By application of said RNAi molecules to cancers in vivo it is possible to effect a treatment whereby the cancer cells are killed or otherwise eliminated.

The invention relates to a method to alter the phenotype of a cell by introducing inhibitory RNA (RNAi) into said cell to ablate mRNAs that encode polypeptides involved in cellular processes.

A number of techniques have been developed in recent years that purport to specifically ablate genes and/or gene products. For example, the use of antisense nucleic acid molecules to bind to and thereby block or inactivate target mRNA molecules is an effective means to inhibit the production of gene products. This is typically very effective in plants where anti-sense technology produces a number of striking phenotypic characteristics. However, recombinant antisense technology is variable leading to the need to screen many, sometimes hundreds of, transgenic organisms carrying one or more copies of an antisense transgene to ensure that the phenotype is indeed truly linked to the antisense transgene expression. Antisense techniques, not necessarily involving the production of stable transfectants, have been applied to cells in culture, with variable results.

In addition, the ability to be able to disrupt genes via homologous recombination has provided biologists with a crucial tool in defining developmental pathways in higher organisms. The use of mouse gene “knock out” strains has allowed the dissection of gene function and the probable function of human homologues to the deleted mouse genes, (Jordan and Zant, 1998).

A much more recent technique to specifically ablate gene function is through the introduction of double stranded RNA, also referred to as inhibitory RNA (RNAi), into a cell which results in the destruction of mRNA complementary to a sequence included in the RNAi molecule. The RNAi molecule comprises two complementary strands of RNA (a sense strand and an antisense strand) annealed to each other to form a double stranded RNA molecule. The RNAi molecule is typically derived from an exonic sequence of the gene that is to be ablated.

Recent studies suggest that RNAi molecules ranging from 100-1000 bp derived from coding sequence are effective inhibitors of gene expression. Surpisingly, only a few molecules of RNAi are required to block gene expression that implies the mechanism is catalytic. The site of action appears to be nuclear as little if any RNAi is detectable in the cytoplasm of cells indicating that RNAi exerts its effect during mRNA synthesis or processing.

The exact mechanism of RNAi action is unknown although there are theories to explain this phenomenon. For example, all organisms have evolved protective mechanisms to limit the effects of exogenous gene expression. For example, a virus often causes deleterious effects on the organism it infects. Viral gene expression and/or replication therefore needs to be repressed. In addition, the rapid development of genetic transformation and the provision of transgenic plants and animals has led to the realisation that transgenes are also recognised as foreign nucleic acid and subjected to phenomena variously called quelling (Singer and Selker, 1995), gene silencing (Matzke and Matzke, 1998), and co-suppression (Stam et. al., 2000).

Initial studies using RNAi used the nematode Caenorhabditis elegans. RNAi injected into the worm resulted in the disappearance of polypeptides corresponding to the gene sequences comprising the RNAi molecule (Montgomery et. al., 1998; Fire et. al., 1998). More recently the phenomenon of RNAi inhibition has been shown in a number of eukaryotes including, by example and not by way of limitation, plants, trypanosomes (Shi et. al., 2000) Drosophila spp. (Kennerdell and Carthew, 2000). Recent experiments have shown that RNAi may also function in higher eukaryotes. For example, it has been shown that RNAi can ablate c-mos in a mouse ooctye and also E-cadherin in a mouse preimplanation embryo (Wianny and Zernicka-Goetz, 2000).

A number of diseases are characterised by uncontrolled cell division, or hyperproliferation, that results in disease. Examples include, psoriasis, cancer and viral diseases that result in cell transformation.

Psoriasis is a generic term to cover a range of diseases characterised by abnormal proliferation of skin cells. The disease covers the following list which is not exhaustive but merely illustrative: nail psoriasis; scalp psoriasis; plaque psoriasis; pustular psoriasis; guttate psoriasis; inverse psoriasis; erythrodermic psoriasis; psoriatic arthritis. Psoriasis is one of the most frequent skin diseases, affecting 1-3% of the Caucasian population world wide. The disease is characterised by alterations in a variety of different cell types. These include epidermal keratinocytes that are characterised by hyperproliferation and an altered differentiation which is indicated by focal parakeratosis and aberrant expression of keratinocyte genes encoding hyperproliferation-associated keratin pair 6/16, involucrin, fillagrin, and integrin adhesion molecules (e.g. VLA-3, 5, 6).

Current methods to control psoriatic conditions include the use of topical applications of coal tar that reduce itching and scaling of skin. However, coal tar sensitises skin to ultraviolet thereby rendering individuals susceptible to sunburn. Treatment with coal tar can also result in photosensitivity. An alternative to the use of coal tar is topical steroids. Although effective, steroid treatment can result in thinning of skin. Also, if steroids are used long term the body can become resistant thereby rendering the treatment ineffective. Other pharmaceutical treatments include the topical application of anthralin, vitamin D3 and retinoid treatment. Oral medications are also available to those with severe forms of the disease that do not respond well to topical treatments. These include methotrexate, cyclosporins, Tegison. Each of these medications has serious side effects which include liver and lung damage (methotrexate), immunosuppression (cyclosporins) and rashes, hair loss and hepatitis (Tegison). Also many of these drugs are incompatible with pregnancy and therefore should be avoided by women of childbearing age. Clearly there is a need for alternative treatments that do not have these disadvantages.

When normal keratinocytes are cultured, they assume a hyperproliferative state that is similar to psoriasis in vivo and has been labelled the pseudo-psoriatic phenotype. This provides an excellent model of testing various therapeutic treatments in vitro before animal model experiments are undertaken Moreover, there exists an animal model for psoriasis that allows the testing of various therapies with respect to the treatment of this condition, see U.S. Pat. No. 5,945,576 which is incorporated by reference. The majority of cells (>95%) that comprise skin are keratinocytes at various stages of differentiation. Keratinocytes of the basal layer are constantly dividing and daughter cells subsequently move outwards, during which they undergo a period of differentiation and arrest cell division. It is the uncontrolled division of these keratinocytes which result in the formation of psoriatic plaques.

Cancer is a further example of uncontrolled cell division. Cancers may arise in many tissues and one idea is that the malignant cells are formed from stem cells of those tissues. Stem cells are cells that have the capacity for self renewal, or differentiation to the characteristic cells of the tissue for which they function as stem cells, or cell death by apoptosis in inappropriate circumstances. Malignancy of the tumour cells that arise from such stem cells may involve both enhanced, uncontrolled cell proliferation and a propensity for self renewal, as well as a reduced propensity for cell death or differentiation.

Teratocarcinomas provide one example of a cancer containing stem cells. These tumours may contain a wide range of differentiated tissues, and have been known in humans for many hundreds of years. They typically occur as gonadal tumours of both men and women, but may also occur extragonadally. The gonadal forms of these tumours are generally believed to originate from germ cells, and the extra-gonadal forms, which typically have the same range of tissues, are thought to arise from germ cells that have migrated incorrectly during embryogenesis. Teratocarcinomas are therefore generally classed as germ cell tumours that encompass a number of different histological types. These include seminoma, embryonal carcinoma, yolk sac carcinoma and choriocarcinoma. Many experiments have shown that EC cells are the stem cells of these tumours and that these EC cells resemble closely cells of the inner cell mass of the early embryo, and embryonic stem (ES) cells derived from such early embryos (Andrews 2002; Henderson et al 2002, Draper et al 2002).

Oct4 is an example of a regulatory protein that is expressed by EC cells and is required for their continued proliferation and self renewal. The elimination of Oct4 protein expression results in the reduction in growth and differentiation of nullipotent and pluripotent human embryonal carcinoma (EC) cells, the malignant counterparts of embryonal stem (ES) cells derived from testicular germ cell tumours, as well as human ES cells themselves. This should allow the malignant cells to be targeted, causing both growth reduction and the differentiation of the malignant cells that make up germ cell tumours. Teratocarcinomas are a form of germ cell tumour, containing Embryonal Carcinoma cells (EC cells). These cells have many features in common with ES/EG cells. The most important of these features is the characteristic of pluripotentiality. The elimination of Oct4 protein expression in germ cell tumours could be achieved by dsRNA against Oct4.

Moreover, in differentiating cultures of embryonic stem cells there is the chance for ES cells to remain undifferentiated as contaminating cells. These cells may present problems at a later stage if the cells are to used for transplantation, due to there pluripotency and increased growth rate relative to the differentiated derivatives which could result in teratocarcinoma formation in recipients. Targeting Oct4 mRNA using RNAi in these cultures would result in the elimination of contaminating cells thus improving the safety of ES cells as a medical treatment. Furthermore the use of dsRNA against Oct4 to render germ cells in animals incapable of producing gametes will make an animal sterile.

It would be desirable to be able to modulate the immune response of stem cells, particularly embryonic stem cells, so that they are tolerated by the host. This would involve delivering dsRNA in vivo to cells to protect against an immune response.

Furthermore, cells treated with dsRNA in vitro against beta-2-microglobulin or type one HLA complex genes would be less immunogenic

According to an aspect of the invention there is provided a method to inhibit cell division comprising introducing at least one inhibitory RNA molecule into a hyperproliferative cell wherein said inhibitory RNA molecule inhibits the expression of at least one gene which mediates at least one essential process in said cell.

In a preferred method of the invention said RNAi molecule inhibits at least one step in the cell-division-cycle of said cell.

The term “hyperproliferative” refers to a cell that shows an uncontrolled cell-division-cycle which can result in a disease condition, for example psoriasis or cancer. It will be apparent to one skilled in the art that the introduction of RNAi molecules into a cell may be by addition of an RNAi molecule to a cell in a composition or by the provision of gene therapy vectors adapted to express both sense and antisense nucleic acids thereby producing an RNAi molecule in situ.

The word “essential” typically refers to genes that are required for i) self-renewal, ii) proliferation iii) inhibition of differentiation iv) inhibition of apoptosis. Inhibiting any one of these will be of value as it would lead to loss of e.g. undifferentiated (malignant) stem cells.

In a yet further preferred method of the invention said hyperproliferative cell is a keratinocyte, preferably a psoriatic keratinocyte.

In a preferred method of the invention said hyperproliferative cell is a cancer cell, preferably a germ cell cancer cell.

In a further preferred method of the invention said hyperproliferative cell is an embryonal carcinoma cell.

In a yet further preferred method of the invention said hyperproliferative cell is an embryonic stem cell.

In a further preferred method of the invention of the invention said sequence comprises at least one of the sequences, or part thereof, which encodes a protein selected from the group consisting of: hyperproliferation-associated keratin pair 6/16, involucrin, fillagrin, integrin adhesion molecules (e.g. VLA-3, 5, 6),

In a preferred method of the invention said RNAi molecule is designed with reference to the nucleic acid sequence shown in any of FIGS. 9-15, or part thereof.

According to a further aspect of the invention there is provided a method to inhibit an immune response comprising introducing at least one inhibitory RNA molecule (RNAi) into a cell wherein said RNAi molecule inhibits the expression of at least one gene which mediates at least one immunogenic response to said cell.

In a preferred method of the invention said cell is a stem cell, preferably said stem cell is embryonic.

In a further preferred method of the invention said HLA gene is beta-2-microglobulin, and is designed with reference to a nucleic acid molecule comprising a nucleic acid sequence as represented in FIG. 17.

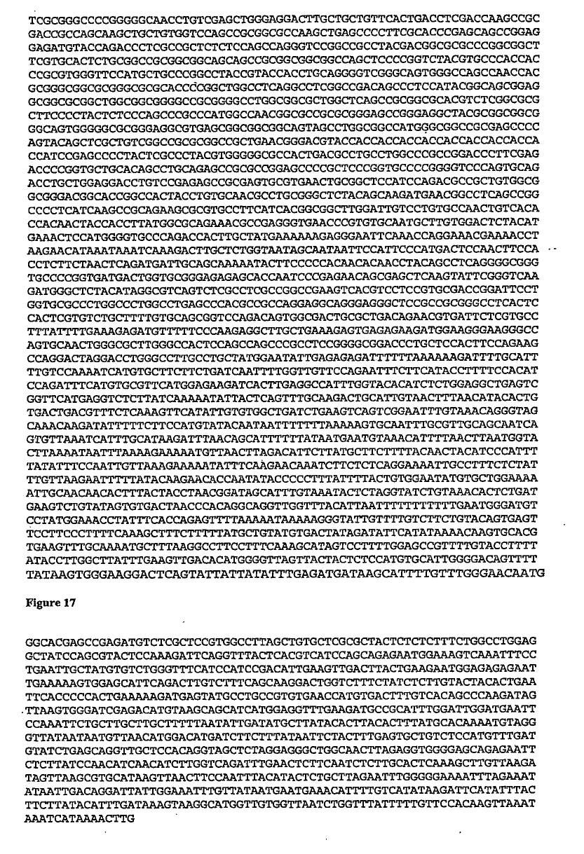

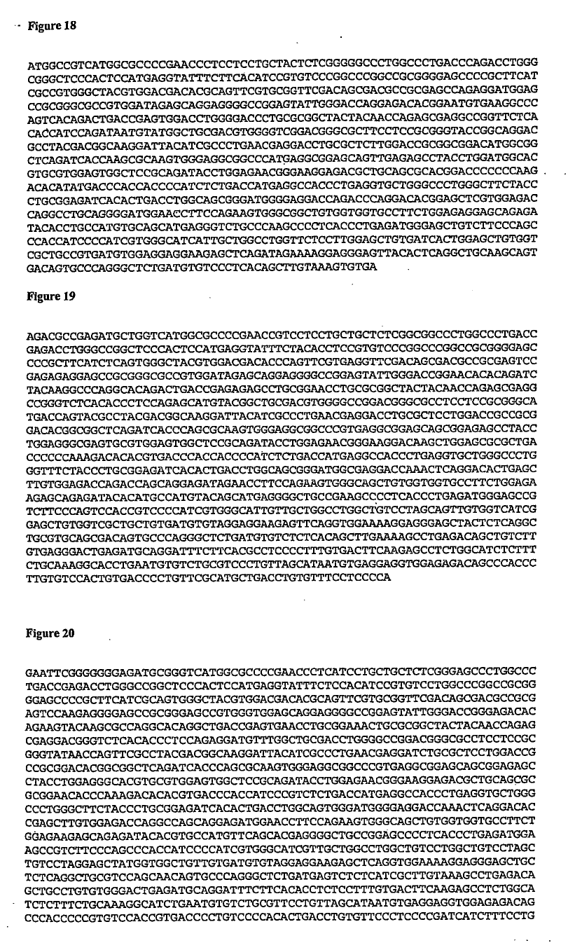

In a still further preferred method of the invention said gene is a type 1 HLA complex gene and is designed with reference to a nucleic acid molecule comprising a nucleic acid sequence as represented in FIG. 18.

In a still further preferred method of the invention said gene is a type 1 HLA complex gene and is designed with reference to a nucleic acid molecule comprising a nucleic acid sequence as represented in FIG. 19.

In a still further preferred method of the invention said gene is a type 1 HLA complex gene and is designed with reference to a nucleic acid molecule comprising a nucleic acid sequence as represented in FIG. 20.

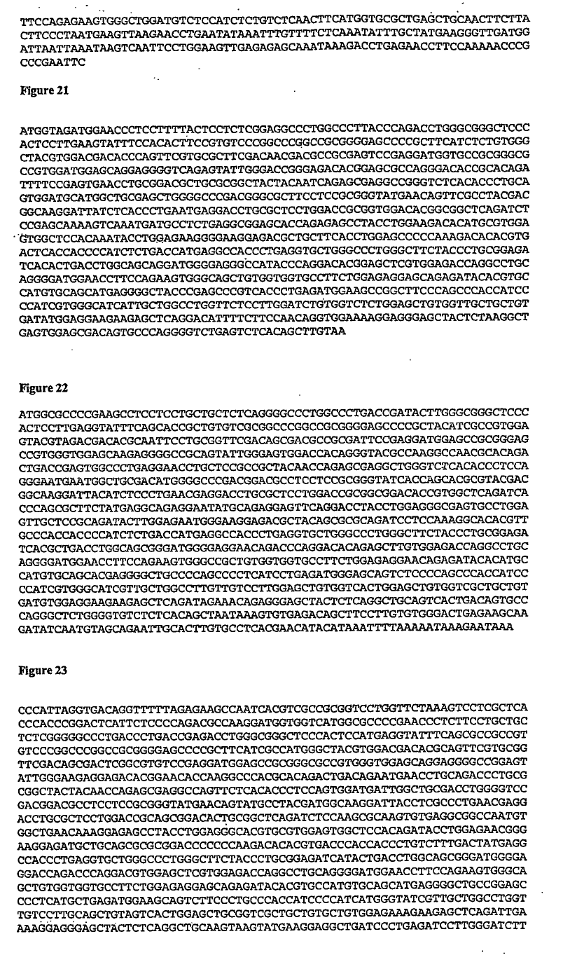

In a still further preferred method of the invention said gene is a type 1 HLA complex gene and is designed with reference to a nucleic acid molecule comprising a nucleic acid sequence as represented in FIG. 21.

In a still further preferred method of the invention said gene is a type 1 HLA complex gene and is designed with reference to a nucleic acid molecule comprising a nucleic acid sequence as represented in FIG. 22.

In a still further preferred method of the invention said gene is a type 1 HLA complex gene and is designed with reference to a nucleic acid molecule comprising a nucleic acid sequence as represented in FIG. 23.

According to a further aspect of the invention there is provided an RNAi molecule characterised in that it comprises the coding sequence of at least one gene that mediates at least one essential process in at least one hyperproliferative cell.

In a preferred embodiment of the invention the essential process is at least one step in the cell-division-cycle.

In a preferred embodiment said coding sequence is an exon.

Alternatively said RNAi molecule is derived from intronic sequences or the 5′ and/or 3′ non-coding sequences which flank coding/exon sequences of genes which mediate said essential process.

In a further preferred embodiment of the invention the length of the RNAi molecule is between 10 bp-1000 bp. More preferably still when the length of RNAi is in the range of 100-1000 bp the length is selected from 100 bp; 200 bp; 300 bp; 400 bp; 500 bp; 600 bp; 700 bp; 800 bp; 900 bp; or 1000 bp. More preferably still said RNAi is at least 1000 bp. More preferably still when the length of RNAi is in the range of 10-1000 bp the length is preferably from 10-100 bp and may be selected from 10 bp; 20 bp; 30 bp; 40 bp; 50 bp; 60 bp; 70 bp; 80 bp; 90 bp; or 100 bp.

In a preferred embodiment of the invention said RNAi molecule is 21 bp.

In yet a further preferred embodiment of the invention said RNAi molecules comprise modified ribonucleotide bases.

It will be apparent to one skilled in the art that the inclusion of modified bases, as well as the natural bases cytosine, uracil, adenosine and guanosine, may confer advantageous properties on RNAi molecules containing said modified bases. For example, modified bases may increase the stability of the RNAi molecule thereby reducing the amount required to produce a desired effect.

In a preferred embodiment of the invention said RNAi molecule comprises at least one nucleic acid sequence presented in FIG. 9-16, or part thereof.

According to a further aspect of the invention there is provided an RNAi molecule characterised in that it comprises the coding sequence of at least one gene that mediates at least one immunogenic response in at least one immune responsive cell.

Preferred embodiments applicable to RNAi molecules which mediate at least one essential process in a hyperproliferative cell are equally applicable to the aspect directly above.

According to a further aspect of the invention there is provided a nucleic acid molecule comprising an expression cassette which cassette comprises a nucleic acid sequence which encodes at least part of a gene that mediates at least one essential process in at least one hyperproliferative cell wherein said cassette is adapted such that both sense and antisense nucleic acid molecules are transcribed from said cassette.

According to a further aspect of the invention there is provided a nucleic acid molecule comprising an expression cassette which cassette comprises a nucleic acid sequence which encodes at least part of a gene that mediates at least one immunogenic response in at least one immune responsive cell wherein said cassette is adapted such that both sense and antisense nucleic acid molecules are transcribed from said cassette.

In a preferred embodiment of the invention said cassette is provided with at least two promoters adapted to transcribe sense and antisense strands of said nucleic acid molecule

In a further preferred embodiment of the invention said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

An alternative embodiment involves the synthesis of so called “stem loop RNAi” molecules that are synthesised from expression cassettes carried in vectors. The DNA molecule encoding the stem-loop RNA is constructed in two parts, a first part that is derived from a gene the regulation of which is desired. The second-part is provided with a DNA sequence which is complementary to the sequence of the first part. The cassette is typically under the control of a promoter that transcribes the DNA into RNA. The complementary nature of the first and second parts of the RNA molecule results in base pairing over at least part of the length of the RNA molecule to form a double stranded hairpin RNA structure or stem-loop. The first and second parts can be provided with a linker sequence. Stem loop RNAi has been successfully used in plants to ablate specific mRNA's and thereby affect the phenotype of the plant, see, Smith et al (2000) Nature 407, 319-320.

In a preferred embodiment of the invention said first and second parts are linked by at least one nucleotide base. In a further preferred embodiment of the invention said first and second parts are linked by 2, 3, 4, 5, 6, 7, 8, 9, or 10 nucleotide bases. In a yet further preferred embodiment of the invention said linker is at least 10 nucleotide bases.

It will be apparent to one skilled in the art that the synthesis of RNA molecules which form RNAi can be achieved by providing vectors which include target genes, or fragments of target genes, operably linked to promoter sequences. Typically, promoter sequences are phage RNA polymerase promoters (e.g. T7, T3, SP6) if RNAi molecules are produced in vitro. Advantageously vectors are provided with multiple cloning sites into which genes or gene fragments can be subcloned. Typically, vectors are engineered so that phage promoters flank multiple cloning sites containing the gene of interest. Phage promoters are oriented such that one promoter synthesises sense RNA and another promoter, antisense RNA. Thus, the synthesis of RNAi is facilitated. Target genes, or fragments of target genes, can be fused directly to promoters to create chimeric promoter/nucleic acid fusions via oligosynthesising technology. Constructs thus created can be easily amplified by polymerase chain reaction to provide templates for the manufacture of RNA molecules.

In a preferred embodiment of the invention said cassette comprises a nucleic acid molecule consisting of a nucleic acid sequence as presented in FIG. 9-16, or part thereof.

In an alternative preferred embodiment of the invention said cassette comprises a nucleic acid molecule consisting of a nucleic acid sequence as presented in any of FIGS. 17-23, or part thereof.

According to an aspect of the invention there is provided a vector comprising a cassette according to the invention.

If it is desired to form RNAi molecules in situ, expression vectors can be adapted to provide expression cassettes comprising nucleic acid molecules under the control of promoter sequences which result in the formation of sense and antisense RNA molecules. This may be facilitated by positioning promoter sequences upstream and downstream of said nucleic acid molecule such that both sense and antisense molecules are transcribed from the vector. Alternatively, separate expression cassettes can be adapted to produce sense and antisense RNAs.

Preferably said further nucleic acid sequence comprises a promoter sequence functional in a specific cell-type. Preferably the promoter sequence is functional in a keratinocyte, ideally a psoriatic cell or psoriatic keratinocyte.

In an alternative preferred embodiment of the invention said promoter sequence is a cancer specific promoter.

Preferably the promoter sequence is functional in an immune responsive cell or an embryonic stem cell or embryonal carcinoma cell. An example of an embryonic stem cell or embryonal carcinoma cell specific promoter is the FGF4 promoter and is disclosed in WO03/080816 that is specifically incorporated by reference. Other examples of promoters with the requisite stem cell specific expression would be known to those skilled in the art and include genes herein disclosed as RNAi target genes.

In a preferred method of the invention said promoter sequences are selected from the following: keratin promoters K1; K5; K6; K10; K14; filaggrin; loricrin; involucurin.

Ideally said promoter sequence is keratin K6. It is known that the K6 promoter shows a high level of expression in epidermal cells undergoing hyperproliferation, see U.S. Pat. No. 5,958,764.

It will be apparent to one skilled in the art that genes that show the requisite expression pattern are readily available. For example, and not by way of limitation, keratin K6, K5 and K14 are described in Woodcock and Mitchell, J. Cell Biol. 95, p580-88 (1982); K1 and K10 are described in Roop et al Proc Natl. Acad. Sci USA, 80, p716-720, (1983) and Schweizer et al, Cell 37, p159-170, (1984). Many of these genes have been cloned and their sequences published, for example, K5, Lersch et al Mol Cell Biol. 8, p486-493, (1988); K14, Marchuk et al Proc. Natl. Acad. Sci. USA, 82, P1609-1613, (1985) and Knapp et al J. Biol. Chem. 262, 938-945, (1987); K1, Steinert et al., J. Biol. Chem. 260, p7142-7149, (1985); K10, Kreig et al J. Biol. Chem. 260, p5867-5870, (1985); K6, Tyner et al Proc. Natl Acad. Sci. USA, 82, 4683-4687, (1985); loricrin, Yoneda et al. J. Biol. Chem. 267(25), 18060-18066.

Moreover, promoter sequences defining the 5′ regions responsible for transcription activation are known, for example see, Tomic et al Cell Reg. 1, p965-973 (K5, K6, K10, K14); Greenhalgh et al, Mol. Carcinogenesis, 7, p99-110, (1993). Methods for transfecting epidermal cells with vectors including epidermal specific promoters are also known as are methods relating to the heterologous expression of polypeptides in epidermal cells, see Morgan et al, Science, 237, p1476-1479, (1987); Teumer et al FASEB, 4, p3245-3250, (1990); Sellheyer et al Proc. Natl. Acad. Sci USA, 90, p5237-5241, (1993). Cell culture methods for keratinocytes are also known, see U.S. Pat. No. 5,968,546.

According to a further aspect of the invention there is provided a method to manufacture RNAi molecules comprising:

(i) providing a vector or chimeric promoter/gene fusion;

(ii) providing reagents and conditions which allow the synthesis of each RNA strand comprising said RNAi molecule; and

(iii) providing conditions which allow each RNA strand to associate over at least part of their length, or at least that part corresponding to the nucleic acid sequence encoding a gene which mediates at least one essential process in a hyperproliferative cell.

According to a further aspect of the invention there is provided a method to manufacture RNAi molecules comprising:

(i) providing a vector or chimeric promoter/gene fusion;

(ii) providing reagents and conditions which allow the synthesis of each RNA strand comprising said RNAi molecule; and

(iv) providing conditions which allow each RNA strand to associate over at least part of their length, or at least that part corresponding to the nucleic acid sequence encoding a gene which mediates at least one immunogenic response in an immune responsive cell.

In vitro transcription of RNA is an established methodology. Kits are commercially available which provide vectors, ribonucleoside triphosphates, buffers, RNase inhibitors, RNA polymersases (eg phage T7, T3, SP6) which facilitate the production of RNA

According to a further aspect of the invention there is provided an in vivo method to inhibit an essential process in a hyperproliferative cell comprising administering to an animal an effective amount of an RNAi according to the invention sufficient to inhibit cell proliferation.

In a preferred method of the invention said inhibition is the inhibition of cancer cell proliferation. Preferably said cancer cell is a teratocarcinoma cell.

Preferably said teratocarcinoma cell is from a cancer selected from the group consisting of: seminoma, embryonal carcinoma, yolk sac carcinoma and choriocarcinoma.

It will be apparent to one skilled in the art that RNAi relies on homology between the target gene RNA and the RNAi molecule. This confers a significant degree of specificity to the RNAi molecule in targeting hyperproliferative cells.

According to a further aspect of the invention there is provided an in vitro method for the treatment of a preparation of cells comprising embryonic stem cells comprising adding to said preparation at least one RNAi molecule according to the invention sufficient to inhibit the proliferation of said embryonic stem cells in said preparation.

In a preferred method of the invention said preparation of cells is a mixed population of embryonic stem cells and differentiated cells.

In a further preferred method of the invention said treated cell preparation is administered to an animal, preferably a human, in need of tissue replacement therapy.

According to a further aspect of the invention there is provided an in vivo method to inhibit an immunogenic response in an immune responsive cell comprising administering to an animal an effective amount of RNAi according to the invention sufficient to inhibit an immune response.

According to a further aspect of the invention there is provided an in vitro method to inhibit an immunogenic response in an immune responsive cell comprising treating said immune responsive cell with an effective amount of RNAi according to the invention sufficient to inhibit an immune response.

RNAi molecules may be encapsulated in liposomes to provide protection from an animals immune system and/or nucleases present in an animals serum.

Liposomes are lipid based vesicles which encapsulate a selected therapeutic agent which is then introduced into a patient. Typically, the liposome is manufactured either from pure phospholipid or a mixture of phospholipid and phosphoglyceride. Typically liposomes can be manufactured with diameters of less than 200 nm, this enables them to be intravenously injected and able to pass through the pulmonary capillary bed. Furthermore the biochemical nature of liposomes confers permeability across blood vessel membranes to gain access to selected tissues. Liposomes do have a relatively short half-life. So called STEALTH® liposomes have been developed which comprise liposomes coated in polyethylene glycol (PEG). The PEG treated liposomes have a significantly increased half-life when administered intravenously to a patient. In addition STEALTH® liposomes show reduced uptake in the reticuloendothelial system and enhanced accumulation selected tissues. In addition, so called immuno-liposomes have been develop which combine lipid based vesicles with an antibody or antibodies, to increase the specificity of the delivery of the RNAi molecule to a selected cell/tissue.

The use of liposomes as delivery means is described in U.S. Pat. No. 5,580,575 and U.S. Pat. No. 5,542,935 which are incorporated by reference. Commercially available delivery means are readily available to the skilled artisan for example, Exgen™, or Superfect™.

Other vehicles to deliver RNAi molecules include viral vectors and plasmids.

The use of viruses or “viral vectors” is well known in the art. A number of viruses are commonly used as vectors for the delivery of exogenous genes. Commonly employed vectors include recombinantly modified enveloped or non-enveloped DNA and RNA viruses, preferably selected from baculoviridiae, parvoviridiae, picornoviridiae, herpesveridiae, poxyiridae, adenoviridiae, or picommaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred. Most preferred are human adenoviral type 5 vectors.

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein an gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al., U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See, e.g. Yoshida and Hamada (1997) Biochem. Biophys. Res. Comm. 230:426-430; Iida, et al. (1996) J. Virol. 70(9):6054-6059; Hwang, et al. (1997) J. Virol 71(9):7128-7131; Lee, et al. (1997) Mol. Cell. Biol. 17(9):5097-5105; and Dreher, et al. (1997) J. Biol. Chem 272(46); 29364-29371. Embryonal stem cell promoters are preferred.

The viruses may also be designed to be selectively replicating viruses. Particularly preferred selectively replicating viruses are described in Ramachandra, et al. PCT International Publication No. WO 00/22137, International Application No. PCT/US99/21452 published Apr. 20, 2000 and Howe, J., PCT International Publication No. WO WO0022136, International Application No. PCT/US99/21451 published Apr. 20, 2000. A particularly preferred selectively replicating recombinant adenovirus is the virus d101/07/309 as more fully described in Howe, J.

It will be apparent to one skilled in the art that the RNAi molecules can be provided in the form of an oral or nasal spray, an aerosol, suspension, emulsion, and/or eye drop fluid. Alternatively the RNAi molecules may be provided in tablet form. Alternative delivery means include inhalers or nebulizers.

According to a yet further aspect of the invention there is provided a therapeutic composition comprising at least one RNAi molecule according to the invention. Preferably said RNAi molecule is for use in the treatment of hyperproliferative diseases.

In a preferred embodiment of the invention said hyperproliferative disease is psoriasis.

In a preferred embodiment of the invention said hyperproliferative disease is cancer.

In a preferred embodiment of the invention said cancer is a disease is a germ cell tumour.

In a preferred embodiment of the invention said cancer is a disease is a teratocarcinoma.

Preferably said cancer is selected from the group consisting of: seminoma, embryonal carcinoma, yolk sac carcinoma and choriocarcinoma.

Preferably said RNAi molecule is for use in the treatment of conditions which would benefit from immunosuppression. More preferably still the condition is transplant rejection.

In a further preferred embodiment of the invention said therapeutic composition further comprises a diluent, carrier or excipient.

An embodiment of the invention will now be described by example only and with reference to the following figures:

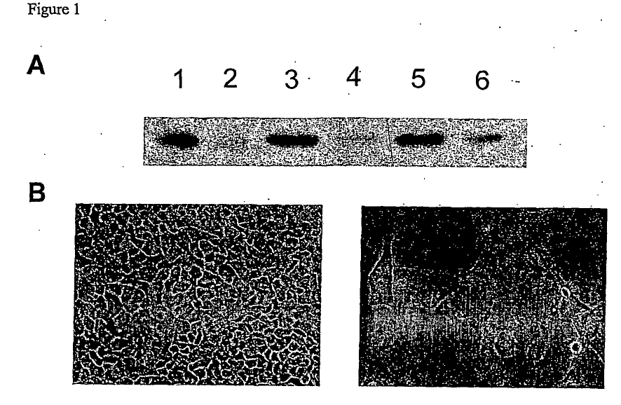

FIG. 1 illustrates: A: Western blot for Oct-4 expression in cells treated with dsRNA targeting either beta2M (Lanes 1, 3, 5) or Oct4 (Lanes 2, 4, 6) after 3 days (Lanes 1, 2), 5 days (Lanes 3, 4) or 7 days (Lanes 5, 6). B: Morphology of 2102Ep cells, 7 days after treating with siRNA targeting beta2M (panel A) and Oct-4 (panel B);

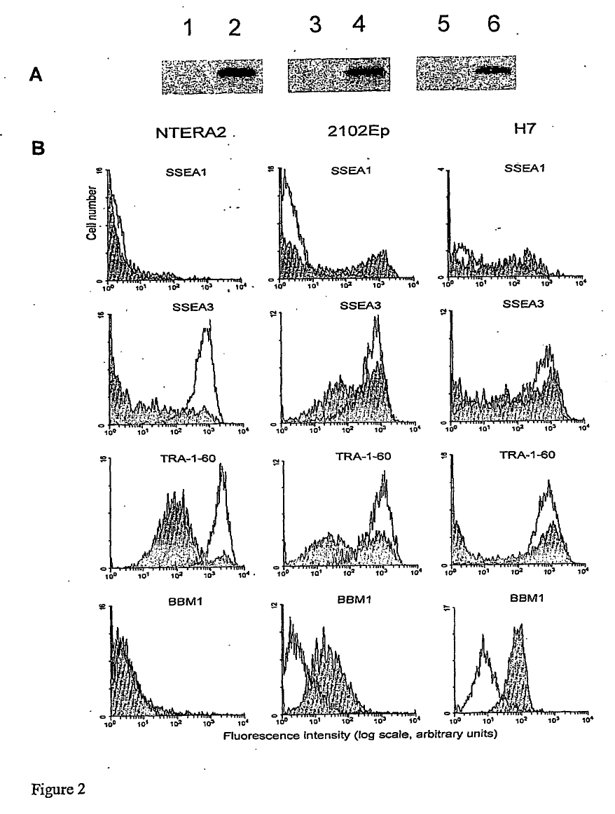

FIG. 2 illustrates: NTERA2 and 2102Ep EC cells and H7 ES cells were treated with dsRNAs targeting Oct-4, and β2M and analysed 5 days later. a, Western blot for Oct-4 protein in NTERA2 (lanes 1, 2), 2102Ep (lanes 3, 4) and H7 (lanes 5, 6); siRNA to Oct-4 (lanes, 1, 3 and 5; siRNA to 2M (lanes 2, 4 and 6). B:Surface antigen expression after treatment with siRNA targeting Oct-4 (shaded histograms) and β2M (non-shaded histograms);

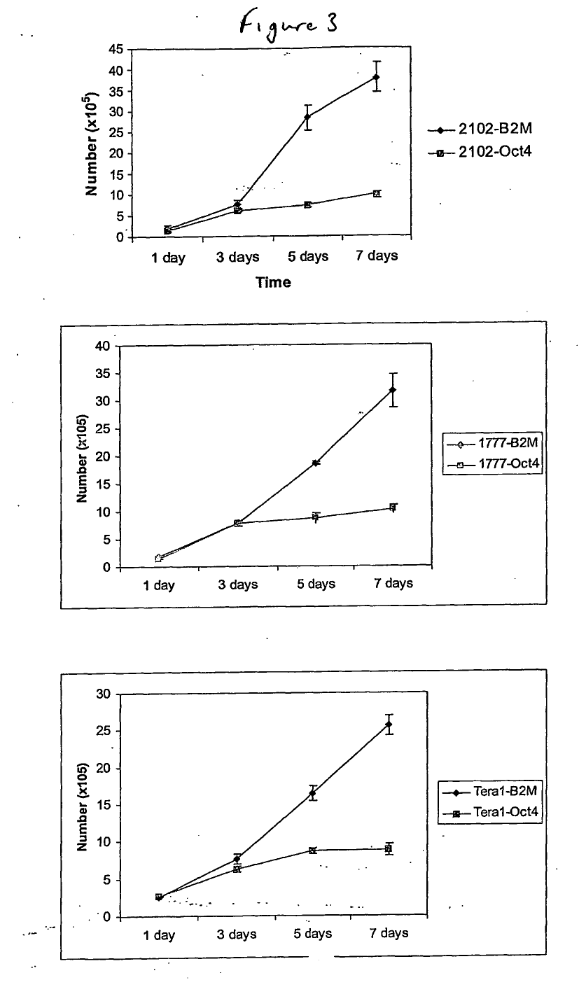

FIG. 3 illustrates: Cells were harvested and counted from triplicate wells that had originally been seeded with 2×10⁵ cells one day before treatment with dsRNA to −b2M and Oct-4 on day 0;

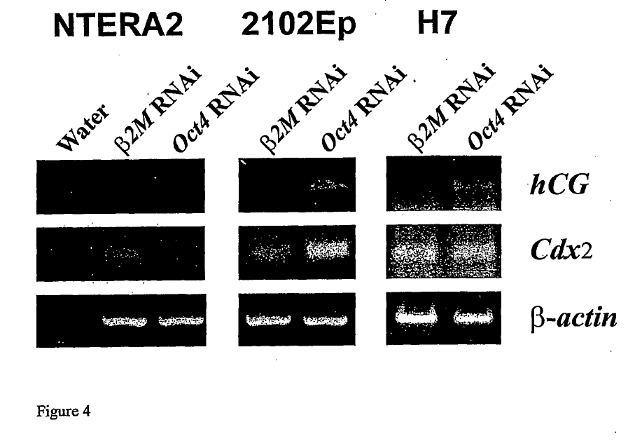

FIG. 4 illustrates: After treatment with dsRNAs targeting Oct4 and β2M, the expression of hCG and Cdx2 were examined by RT-PCR. β-actin PCR was used as a template loading control. In response to Oct-4 RNAi, the trophoblast-specific mRNA, hCG, was induced in 2102Ep and H7 cells, and Cdx2, which encodes a transcription factor associated with trophectoderm differentiation, was up-regulated in 2102Ep but down-regulated in NTERA2 cells. Cdx2 was expressed appreciably in control H7 cells and no significant change was seen after treatment with siRNA to Oct-4;

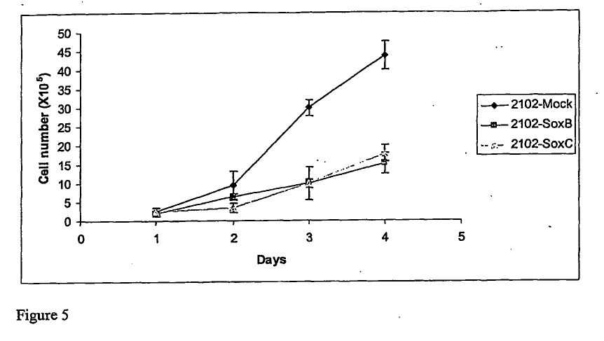

FIG. 5: Reduction in cell number following the treatment of 2102Ep cells with 2 different siRNA's against Sox2 (SoxB and SoxC) compared with mock (no siRNA) treated cells;

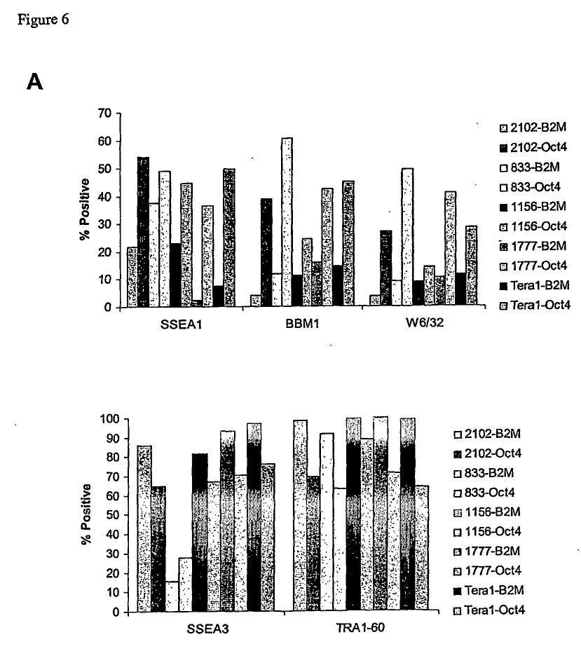

FIG. 6: Flow cytometric analysis of cell surface antigens expressed on human EC and ES cells after treatment with dsRNA against beta-2-microglobulin (β2M) and Oct4. Panel A: percentage positive cells. Panel B mean fluorescence.

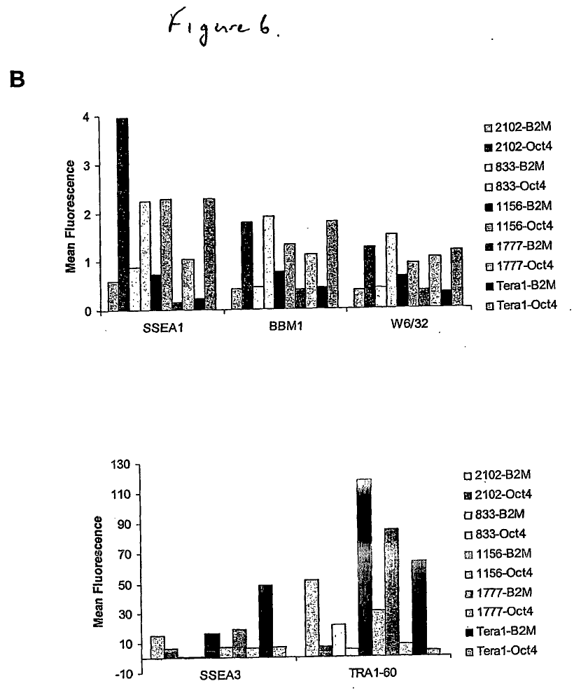

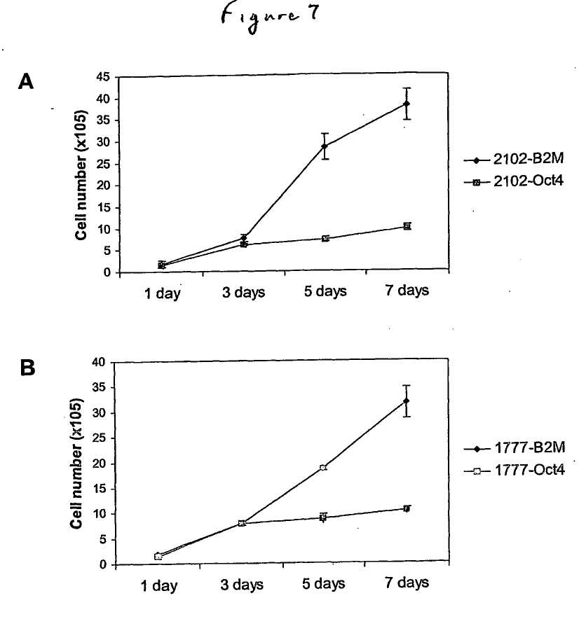

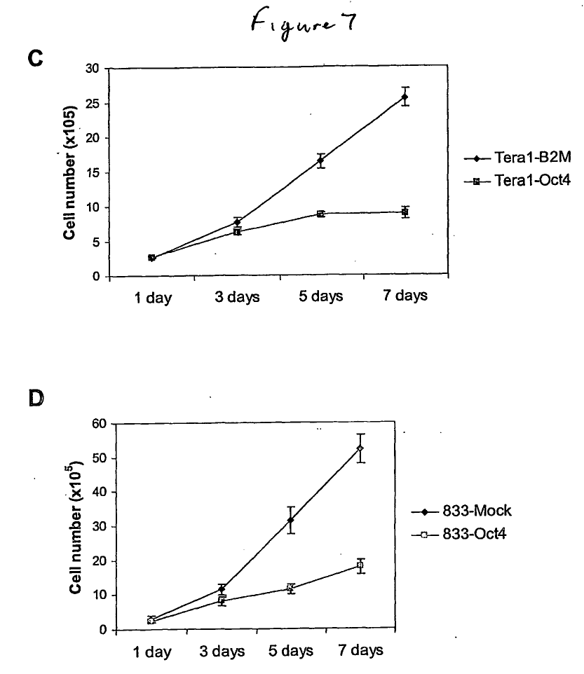

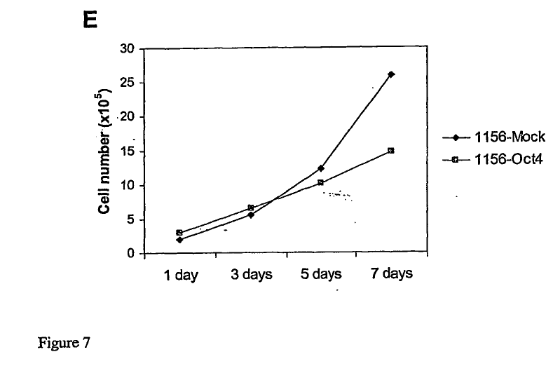

FIG. 7: Reduction in cell growth in a range of human embryonal carcinoma cells lines treated with either Mock (no dsRNA, diamonds) or siRNA to Oct4 (squares). A:2102Ep, B:1777N, C: Tera-1, D: 833KE, E: 1156QE.;

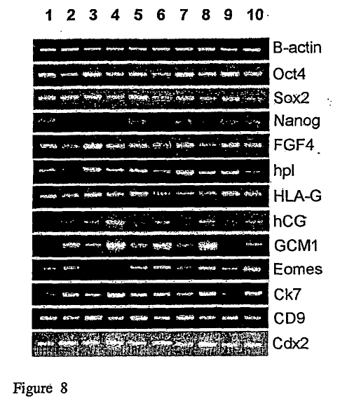

FIG. 8: RT-PCR analysis of embryonal carcinoma cells treated with siRNA to beta-2-microglobulin (β2M) or Oct4. Lanes from left to right; 1: 2102Ep+B2M siRNA, 2: 2102Ep+Oct4 siRNA, 3:833KE+B2M siRNA, 4: 833KE+ Oct4 siRNA, 5: 1156QE+B2M siRNA, 6: 1156QE+ Oct4 siRNAp; 7: 1777Nrpmet+B2M siRNA, 8:1777Nrpmat+Oct4 siRNA, 9: Tera-1+B2M siRNA, 10:Tera-1+Oct4 siRNA;

FIG. 9 is the nucleic acid sequence of Oct 4;

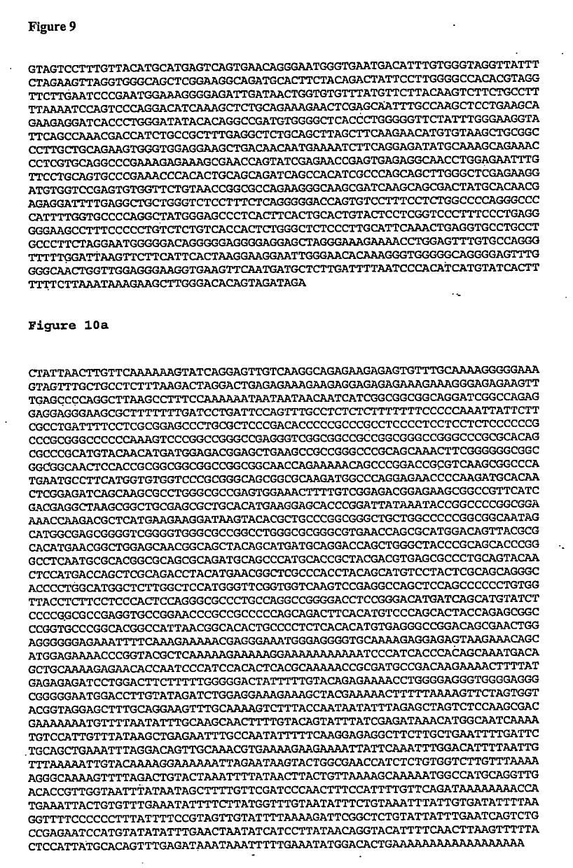

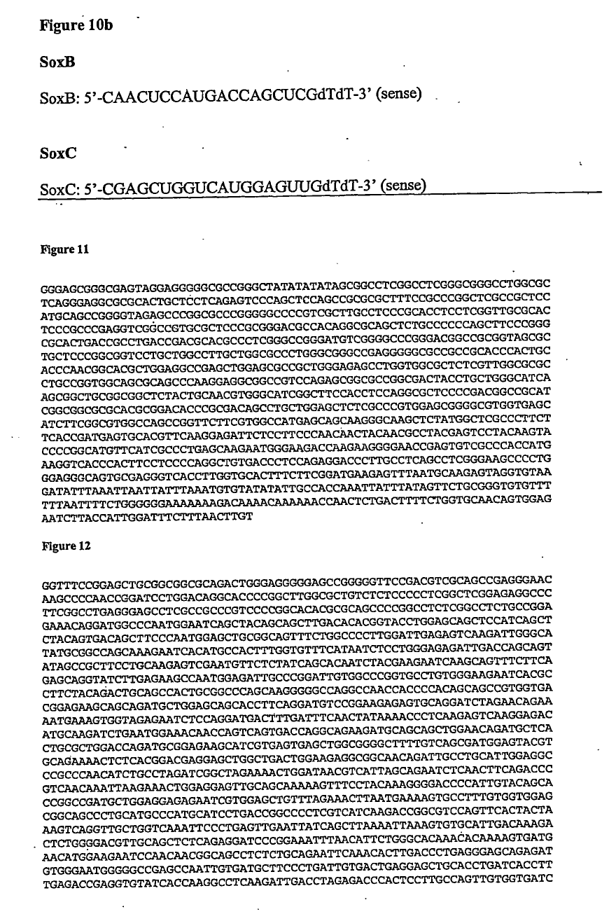

FIG. 10A is the nucleic acid sequence of Sox 2; FIG. 10B 21 bp RNAi molecules derived from Sox

FIG. 11 is the nucleic acid sequence of FGF4;

FIG. 12 is the nucleic acid sequence of STAT3;

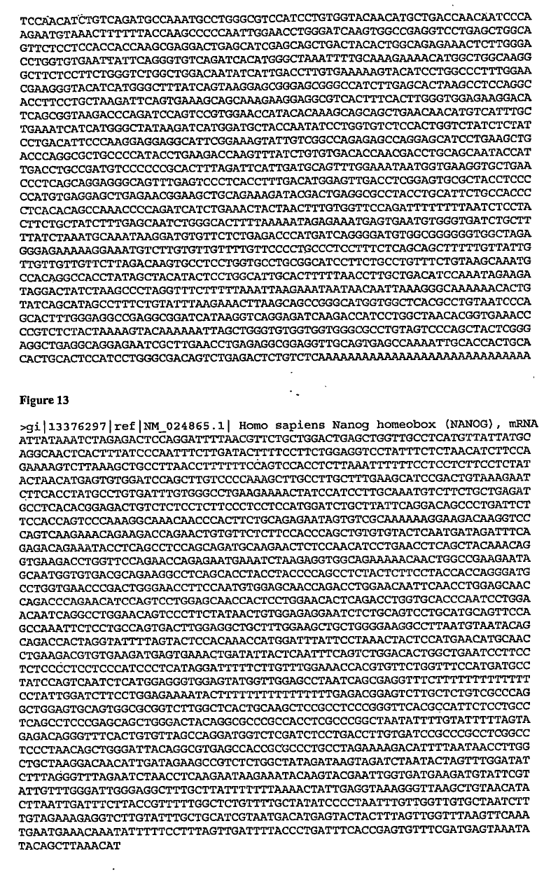

FIG. 13 is the nucleic acid sequence of NANOG;

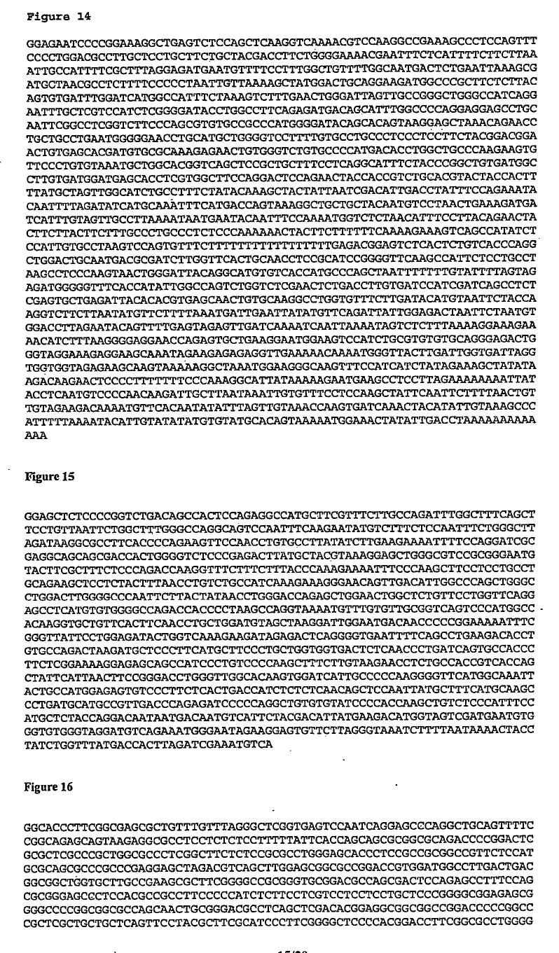

FIG. 14 is the nucleic acid sequence of TDGF;

FIG. 15 is the nucleic acid sequence of GDF3;

FIG. 16 is the nucleic acid sequence of GATA 6;

FIG. 17 is the nucleic acid sequence of Beta-2-microglobulin;

FIG. 18 is the nucleic acid sequence of HLA-A;

FIG. 19 is the nucleic acid sequence of HLA-B;

FIG. 20 is the nucleic acid sequence of HLA-C;

FIG. 21 is the nucleic acid sequence of HLA-E;

FIG. 22 is the nucleic acid sequence of HLA-F;

FIG. 23 is the nucleic acid sequence of HLA-G; and

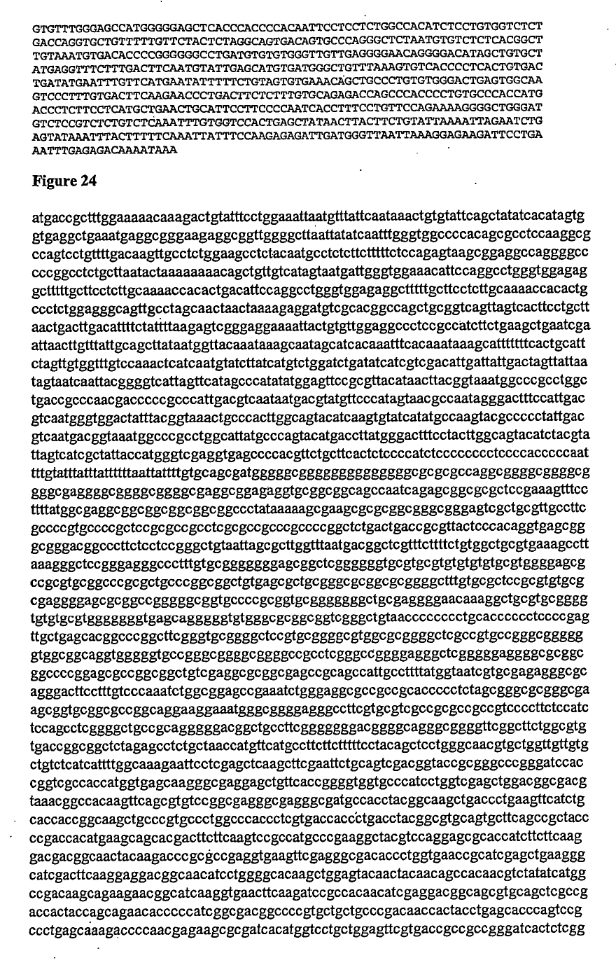

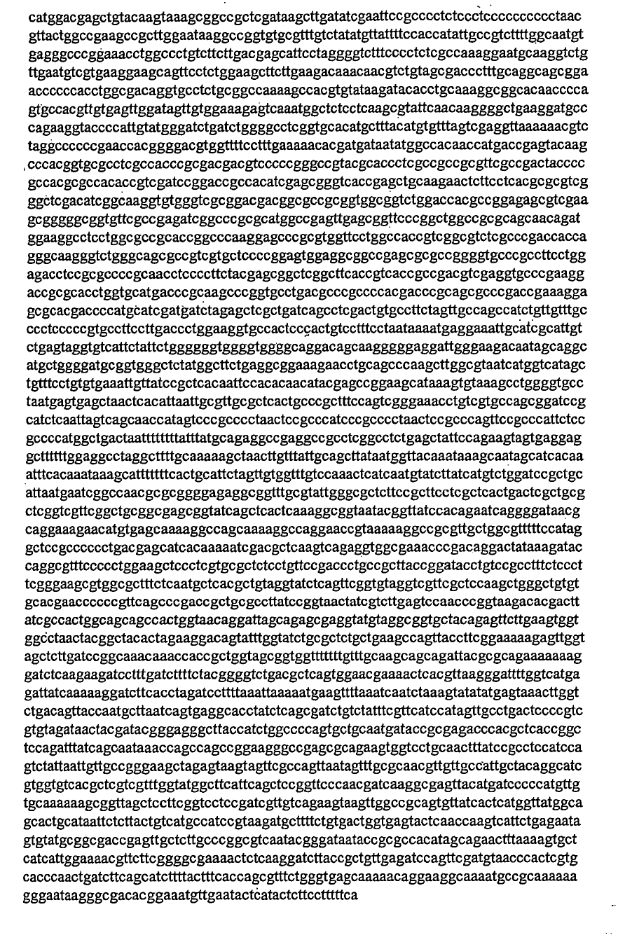

FIG. 24 is the nucleic acid sequence of pCAG-GFP

### MATERIALS AND METHODS

Cell Culture Conditions

Normal human keratinocytes (NHK) were isolated from skin after a punch biopsy and were then maintained in Keratinocyte-SFM.

NTERA2 c1.Dl and 2102Ep c1.2A6 human EC cells were cultured in DMEM containing 10% heat-inactivated FCS, as previously described. The human ES cell line, H7 (ref 1) was cultured in ‘Knock-Out’-DMEM (Invitrogen) supplemented with 20% Serum Replacement (Invitrogen) and 4 ng/ml bFGF (Invitrogen), on feeder layers of mouse embryonic fibroblasts mitotically inactivated with Mitomycin C^{1,16}. For stable transfection, the cells were seeded at 3×10⁴ per cm² and transfected one day later with pCAG-GFP using Exgen 500 (Fermentas) following the manufacturer's protocol. Puromycin (1 μg/ml) was added to the cultures, 24 hours after transfection, and puromycin-resistant green-fluorescent colonies were selected and expanded for further experiments.

RNAi Production and Treatment of Keratinocytes

DNA fragments corresponding to specific gene sequences were amplified by PCR (Mullis and Faloona, 1987) to generate specific templates for RNAi production. Both primers used in a particular reaction contained a 5′ T7 RNA polymerase binding site (TAATACGACTCACTATAGGGAGA) appended to gene specific sequences. PCR-reactions were electrophoresed in 1% agarose and the single band at the correct size for the amplification was excised and purified. 5 μl of a 100 μl PCR reaction was used as a template for a MEGASCRIPT T7 transcription reaction (Ambion, USA). The products from the MEGASCRIPT reaction were precipitated and re-suspended in water. The RNAi was created by heating the MEGASCRIPT reactions to 65° C. for 30 minutes and cooling to room temperature to allow annealing. To test for production of full length dsRNA 6 μg was electrophoresed on a 1% denaturing agarose gel and stained with ethidium bromide. The RNA was observed by transillumination with 302 nm UV light.

The following method describes RNAi of cells cultured in 6 well plates. Volumes and cell numbers should be scaled appropriately for larger or smaller culture vessels.

Cells were seeded at 500,000 per well on the day prior to treatment and grown in their normal medium. For each well to be treated, 9.5 μg of the double stranded RNA of interest was diluted in 300 μl of 150 mM NaCl. 21 μl of ExGen 500 (MBI Fermentas) was added to the diluted RNA solution and mixed by vortexing. The dsRNA/ExGen 500 mixture was incubated at room temperature for 10 minutes. 3 ml of fresh cell growth medium was then added, producing the RNAi treatment medium. Growth medium was aspirated from the culture vessel and replaced with 3 ml of RNAi treatment medium per well. Culture vessels were then centrifuged at 280 g for 5 minutes and returned to the incubator. After 12-18 hrs, RNAi treatment medium was replaced with normal growth medium and the cells maintained as required.

SiRNA Transfection Protocol for Oligofectamine]

Cells were trypsinised and plated in 6 well plates at about 2×10⁴ per cm² (they should be 30-50% confluent at the time of transfection). The next day, siRNA was introduced into the cells using the Oligofectamine transfection reagent (Invitrogen) following the manufacturer's protocol. In brief, 10 μl siRNA (20 μM solution) was incubated with 4 μL Oligofectamine in 190 μl Optimem (Invitrogen) for 20 min; the mixture was then added to the cells in a final volume of 1.2 ml. The transfected cells were cultured and were fed daily with fresh medium until assayed.

RNAi Production and Treatment of EC and ES Cells

siRNAs corresponding to eGFP, μ2M and Oct4 were designed with the following sense and antisense sequences and were synthesized by Xeragon Inc. (Huntsville, USA).

| | | |
|---|---|---|
| eGFP: | 5′-CGUAAACGGCCACAAGUUCdTdT-3′ | (sense) and, |
| | 5′-GAACUUGUGGCCGUUUACGdTdT-3′ | (antisense); |
| β2M: | 5′-GAUUCAGGUUUACUCACGUdTdT-3′ | (sense) and, |
| | 5′-ACGUGAGUAAACCUGAAUCdTdT-3′ | (antisense); |
| Oct4: | 5′-AGCAGCUUGGGCUCGAGAAdTdT-3′ | (sense) and, |
| | 5′-UUCUCGAGCCCAAGCUGCUdTdT-3′ | (antisense). |

Cells were trypsinised and plated in 6 well plates at 2×10⁴ per cm². The next day, siRNA was introduced into the cells using the Oligofectamine transfection reagent (Invitrogen) following the manufacturer's protocol. In brief, 10 μl siRNA (20 μM solution) was incubated with 4 μl Oligofectamine in 190 μl Optimem (Invitrogen) for 20 min; the mixture was then added to the cells in a final volume of 1.2 ml. The transfected cells were cultured and were fed daily with fresh medium until assayed.

RNA Extraction

Growing cultures of cells were aspirated to remove the DME and foetal calf serum. Trace amounts of foetal calf serum was removed by washing in Phosphate-buffered saline. Fresh PBS was added to the cells and the cells were dislodged from the culture vessel using acid washed glass beads. The resulting cell suspension was centrifuged at 300×g. The pellets had the PBS aspirated from them. Tri reagent (Sigma, USA) was added at 1 ml per 10⁷ cells and allowed to stand for 10 mins at room temperature. The lysate from this reaction was centrifuged at 12000×g for 15 minutes at 4° C. The resulting aqueous phase was transferred to a fresh vessel and 0.5 ml of isopropanol/ml of trizol was added to precipitate the RNA. The RNA was pelleted by centrifugation at 12000×g for 10 mins at 4° C. The supernatant was removed and the pellet washed in 70% ethanol. The washed RNA was dissolved in DEPC treated double-distilled water.

Analysis of Keratinocyte Transfectants by Antibody Staining and FACS

Cells were treated with trypsin (0.25% v/v) for 5 mins to disaggregate the cells; they were washed and re-suspended to 2×10⁵ cells/ml. This cell suspension was incubated with 50 μl of primary antibody in a 96 well plate on a rotary shaker for 1 hour at 4° C. Supernatant from a myeloma cell line P3X63Ag8, was used as a negative control. The 96 well plate was centrifuged at 100 rpm for 3 minutes. The plate was washed 3 times with PBS containing 5% foetal calf serum to remove unbound antibody. Cell were then incubated with 50 μl of an appropriate FITC-conjugated secondary antibody at 4° C. for 1 hour. Cells were washed 3 times in PBS+5% foetal calf serum and analysed using an EPICS elite ESP flow cytometer (Coulter eletronics, U.K). (Andrews et. al., 1982).

Analysis of Antigen Expression on EC and ES Cells by Immunofluorescence and FACS

Antigen expression was assayed by immunofluorescence and flow cytometry as previously described (Draper et. al., 2002 using the following monoclonal antibodies: MC631, anti-stage specific embryonic antigen-3 (SSEA3); MC813-70, anti-stage specific embryonic antigen-4 (SSEA4); MC480, anti-stage specific embryonic antigen-1 (SSEA1); TRA-1-60; TRA-2-54, anti-liver/kidney/bone alkaline phosphatase; BBM1; W6/32.

Northern Blot Analysis of RNA Extracted from Keratinocytes

RNA separation relies on the generally the same principles as standard DNA but with some concessions to the tendancy of RNA to hybridise with itself or other RNA molecules. Formaldehyde is used in the gel matrix to react with the amine groups of the RNA and form Schiff bases. Purified RNA is run out using standard agarose gel electrophoresis. For most RNA a 1% agarose gel is sufficient. The agarose is made in 1×MOPS buffer and supplemented with 0.66M formaldehyde. Dried down RNA samples are reconstituted and denatured in RNA loading buffer and loaded into the gel. Gels are run out for apprx. 3 hrs (until the dye front is ¾ of the way down the gel).

The major problem with obtaining clean blotting using RNA is the presence of formaldehyde. The run out gel was soaked in distilled water for 20 mins with 4 changes, to remove the formaldehyde from the matrix. The transfer assembly was assembled in exactly the same fashion as for DNA (Southern) blotting. The transfer buffer used however was 10× SSPE. Gels were transferred overnight. The membrane was soaked in 2× SSPE to remove any agarose from the transfer assembly and the RNA was fixed to the membrane. Fixation was achieved using short-wave (254 nM) UV light. The fixed membrane was baked for 1-2 hrs to drive off any residual formaldehyde.

Hybridisation was achieved in aqueous phase with formamide to lower the hybridisation temperatures for a given probe. RNA blots were prehybridised for 2-4 hrs in northern prehybridisation solution. Labelled DNA probes were denatured at 95° C. for 5 mins and added to the blots. All hybridisation steps were carried out in rolling bottles in incubation ovens. Probes were hybridised overnight for at least 16 hrs in the prehybridisation solution. A standard set of wash solutions were used.

Stringency of washing was achieved by the use of lower salt containing wash buffers. The following wash procedure is outlined as follows

| | | |
|---|---|---|
| 2× SSPE | 15 mins | room temp |
| 2× SSPE | 15 mins | room temp |
| 2× SSPE/0.1% SDS | 45 mins | 65° C. |
| 2× SSPE/0.1% SDS | 45 mins | 65° C. |
| 0.1× SSPE | 15 mins | room temp |

Preparation of Radiolabelled DNA Probes

The method of Feinberg and Vogelstein (Feinberg and Vogelstein, 1983) was used to radioactively label DNA. Briefly, the protocol uses random sequence hexanucleotides to prime DNA synthesis at numerous sites on a denatured DNA template using the Klenow DNA polymerase I fragment. Pre-formed kits were used to aid consistency. 5-100 ng DNA fragment (obtained from gel purification of PCR or restriction digests) was made up in water, denatured for 5 mins at 95° C. with the random hexamers. The mixture was quench cooled on ice and the following were added,

5 μl[α-32P] dATP 3000 Ci/mmol

1 μl of Klenow DNA polymerase (4 U)

The reaction was then incubated at 37° C. for 1 hr. Unincorporated nucleotide were removed with spin columns (Nucleon Biosciences).

Production of cDNA from Keratinocytes

The enzymatic conversion of RNA into single stranded cDNA was achieved using the 3′ to 5′ polymerase activity of recombinant Moloney-Murine Leukemia Virus (M-MLV) reverse transcriptase primed with oligo (dT) and (dN) primers. For Reverse Transcription-Polymerase Chain Reaction, single stranded cDNA was used. cDNA was synthesised from 1 μg poly (A)+ RNA or total RNA was incubated with the following

| | | |
|---|---|---|
| 1.0 | μM | oligo(dT) primer for total RNA or random hexamers for |
| | | mRNA |
| 0.5 | mM | 10 mM dNTP mix |
| 1 | U/μl | RNAse inhibitor (Promega) |
| 1.0 | U/μl | M-MLV reverse transcriptase in manufacturers supplied |
| | | buffer |

(Promega)

The reaction was incubated for 2-3 hours at 42° C.

Fluorescent Automated Sequencing

To check the specificity of the PCR primers used to generate the template used in RNAi production automatic sequencing was carried out using the prism fluorescently labelled chain terminator sequencing kit (Perkin-Elmer) (Prober et al 1987). A suitable amount of template (200 ng plasmid, 100 ng PCR product), 10 μM sequencing primer (typically a 20mer with 50% G-C content) were added to 8 μl of prism pre-mix and the total reaction volume made up to 20 μl. 24 cycles of PCR (94° C. for 10 seconds, 50° C. for 10 seconds, 60° C. for 4 minutes). Following thermal cycling, products were precipitated by the addition of 2 μl of 3M sodium acetate and 50 μl of 100% ethanol. DNA was pelleted in an Eppendorf microcentrifuge at 13000 rpm, washed once in 70% ethanol and vacuum dried. Samples were analysed by the in-house sequencing Service (Krebs Institute). Dried down samples were resuspended in 4 μl of formamide loading buffer, denatured and loaded onto a ABI 373 automatic sequencer. Raw sequence was collected and analysed using the ABI prism software and the results were supplied in the form of analysed histogram traces.

Reverse Transcription and Polymerase Chain Reaction for Determining the Expression of genes in EC and ES cells.

Total RNA (2 μg) was reverse transcribed using 1 μg oligo-dT primer with MMLV Reverse-Transcriptase (Promega) in a 40 μl reaction volume containing 1.25 mM dNTPs at 37° C. Oligonucleotide primers for use in PCR were designed using the PrimerSelect program from the DNASTAR software package (DNASTAR Inc., Madison, Wis.). PCR was performed using 1 μl of cDNA in 25 μl PCR containing 15 pmol of each primer, 0.1 mM dNTPs and 0.3 units Taq polymerase (Promega). Primer sequences used and conditions of these reactions were as follows:

hCG-f:
5′-ATGGGCGGGACATGGGCATCCA-3′, (70° C. annealing,
×35 cycles);
hCG-r:
5′-GGCCCCGGGAGTCGGGATGG-3′, (70° C. annealing,
×35 cycles);
Cdx2-f:
5′-CCTCCGCTGGGCTTCATTCC-3′, (60° C. annealing,
×30 cycles);
Cdx2-r:
5′-TGGGGGTTCTGCAGTCTTTGGTC-3′ (60° C. annealing,
×30 cycles);
β-actin-f:
5′-ATCTGGCACCACACCTTCTACAATGAGCTGCG-3′ (70° C.
annealing, ×25 cycles);
,β-actin-r:
5′-CGTCATACTCCTGCTTGCTGATCCACATCTGC-3′ (70° C.
annealing, ×25 cycles).

Detection of Specific Protein Targets on Keratinocytes by SDS-PAGE and Western Blotting

To obtain cell lysates monolayers of cells were rinsed 3 times with ice-cold PBS supplemented with 2 mM CaCl₂. Cells were incubated with 1 ml/75 cm² flask lysis buffer (1% v/v NP40, 1% v/v DOC, 0.1 mM PMSF in PBS) for 15 min at 4° C. Cell lysates were transferred to eppendorf tubes and passed through a 21 gauge needle to shear the DNA. This was followed by freeze thawing and subsequent centrifugation (30 min, 4° C., 15000 g) to remove insoluble material. Protein concentrations of the supernatants were determined using a commercial protein assay (Biorad) and were adjusted to 1.3 mg/ml. Samples were prepared for SDS-PAGE by adding 4 times Laemmli electrophoresis sample buffer and boiling for 5 min. After electrophoresis with 16 μg of protein on a 10% polyacrylamide gel (Laemmli, 1970) the proteins were transferred to nitro-cellulose membrane with a pore size of 0.45 μm. The blots were washed with PBS and 0.05% Tween (PBS-T). Blocking of the blots occurred in 5% milk powder in PBS-T (60 min, at RT). Blots were incubated with the appropriate primary antibody. Horseradish peroxidase labelled secondary antibody was used to visualise antibody binding by ECL (Amersham, Bucks., UK). Materials used for SDS-PAGE and western blotting were obtained from Biorad (California, USA) unless stated otherwise.

Detection of Specific Protein Targets on EC and ES by SDS-PAGE and Western Blotting

Cells were harvested using trypsin and lysed in RIPA buffer (1× PBS, 1% Nonidet P-40, 0.5% sodium deoxycholate, 0.1% SDS) at 10⁷ cells/ml. These lysates were electrophoresed using SDS-PAGE on a 10% polyacrylamide gel and blotted overnight onto PVDF membrane. Membranes were stained with Ponceau-S to check loading, and blocked for 1 hr with 5% fat-free milk solution. Samples were probed with a goat polyclonal antibody against Oct4 (Santa-Cruz Biotechnology) at a concentration of 0.25 μg/ml and a 1:4000 Dilution of anti-goat IgG-peroxidase conjugate (Sigma-Aldrich). Staining was visualized using ECL kit (Amersham Biosciences).

### EXAMPLES

The Effect of RNAi on Oct4 Protein Expression and on Levels of Cell Proliferation and Differentiation.

The nullipotent EC line, 2102Ep, was treated with siRNA targeting Oct4. The levels of Oct4 protein fell markedly as detected by Western blotting (FIG. 1a). The maximum effect on Oct4 protein level was seen between 3 and 5 days after treatment with siRNA, and the level began to rise again by 8 days. β2-microglobulin siRNA, used as a control, had no effect on Oct4 mRNA and protein level.

Cells treated with Oct4 siRNA grew considerably more slowly (FIG. 3) and acquired a large flat morphology indicative of differentiation (FIG. 1b).

To test the effect of RNAi-mediated knock down of Oct4 expression, 2102Ep and NTERA2 human EC, and H7 human ES cells were treated with siRNA targeting Oct4 and β2M for 3 to 5 days. A marked, specific reduction in Oct4 protein level was observed in each cell line (FIG. 2a) and significant populations of SSEA3-negative and TRA-1-60 negative cells appeared (FIG. 2b). Loss of these characteristic markers of human EC and ES cells is an early indication of differentiation. The appearance of these antigen-negative cells was most obvious in the Oct4 siRNA-treated EC lines, because their cultures showed less tendency to differentiate spontaneously in the controls compared to the ES cells, cultures of which typically contained significant numbers of spontaneously differentiated cells even under optimal conditions. Nevertheless, it was clear that Oct4 RNAi induced the same changes in the ES cells as in the EC cells. At the same time, the number of SSEA1-positive cells, also indicative of differentiation, increased in the 2102Ep EC and H7 ES cultures treated with Oct4 siRNA, although, only a limited appearance of SSEA1-positive cells was noted in the NTERA2 cultures. Again, as expected, β2M siRNA caused a marked down-regulation of β2M expression in 2102Ep and H7 cells compared with cells treated with Oct4 siRNA. In this experiment, little β2-microglobulin expression was seen in the NTERA2 cultures in either case, but this accords with past experience that NTERA2 cells frequently express only very low, barely detectable levels of β2M and HLA, whereas expression of both is regularly observable in 2102Ep and H7 cells.

Exposure of malignant EC cells, 2102, 1777 and Tera-1 to Oct4 RNAi caused a marked reduction in cell growth (FIG. 3). [Two different siRNA's against the pluripotent stem cell marker Sox2 (FIG. 10b) we applied to 2102Ep EC cells. The resultant cells displayed a marked reduction in growth characteristics when compared to the control cells.]

Oct4 siRNA treatment, hCG mRNA was significantly up-regulated in 2102Ep and H7 cells (FIG. 4) indicating that differentiation into trophectoderm is a consequence of lowered Oct4 levels in human EC and ES cells. Consistent with this, the transcription factor Cdx2, associated with this differentiation lineage in the mouse (Rossant and Croos, 2001), was also up-regulated in 2102Ep cells FIG. 4). However, Cdx2 was already expressed at significant levels in the control H7 cells, and no further change was noted after siRNA treatment. This probably reflects the tendency of these pluripotent cells to differentiate spontaneously to some extent in the control cultures. By contrast, NTERA2 cells behaved differently. Indeed, a weak and barely detectable band corresponding to hCG did appear in the cells treated with Oct4 siRNA but, although Cdx2 was expressed in the control cells, it was down-regulated following treatment with Oct4 siRNA. Nevertheless, growth of NTERA2 EC cells was also substantially inhibited. A range of EC cell lines where treated with siRNA to either beta-2-microglobulin or Oct4 as previously described. All the cell lines treated with siRNA to Oct4 showed downregulation of both percent positive cells (FIG. 6A) or the mean florescence of the whole population (FIG. 6B) when compared to the beta-2-microglobulin treated cells. This is consistent with the Oct4 siRNA inducing cellular differentiation in the EC cells.

Treatment of the Cells with siRNA to β2M

Treatment of the cells with siRNA to β2M resulted in a substantial reduction in the binding of antibody BBM1 to μ2-microglobulin and the HLA-A,B,C heavy chain which requires μ2-microglobulin for cell surface expression (Arce-Gomez et. al., 1978) as analysed by flow cytometry.

### REFERENCES

Draper, J. S., Pigott, C., Thomson, J. A. & Andrews, P. W. Surface antigens of human embryonic stem cells: changes upon differentiation in culture. J. Anat. 200, 249-58 (2002).
Rossant, J. & Cross, J. C. Placental development: lessons from mouse mutants. Nat. Genet. Rev. 2, 538-548 (2001).
Arce-Gomez, B., Jones, E. A., Barnstable, C. J., Solomon, E. & Bodmer, W. F. The genetic control of HLA-A and B antigens in somatic cell hybrids: requirement for beta2 microglobulin. Tissue Antigens 11, 96-112 (1978).
Henderson, J., Draper, J. S., Baillie, H. S., Fishel, S., Thomson, J. A., Moore, H., Andrews, P. W. (2002) Pre-implantation human embryos and embryonic stem cells show comparable expression of stage specific embryonic antigens. Stem Cells, 20: 329-337.
Andrews, P. W. (2002). From teratocarcinomas to embryonic stem cells. Phil. Trans. R. Soc. Lond. B357, 405-417.

1. A method to inhibit cell division of a cancer cell comprising introducing at least one inhibitory RNA molecule designed with reference to the nucleic acid sequence shown in any of FIGS. 9-16 into said cancer cell wherein said inhibitory RNA molecule inhibits the expression of at least one gene which mediates at least one essential process in said cell.

2. A method according to claim 1 wherein said RNAi molecule inhibits at least one step in the cell-division-cycle of said cell.

3. A method according to claim 1 wherein said cell is a germ cell carcinoma cell, particularly a teratocarcinoma cell.

4. (canceled)

5. An RNAi molecule comprising at least one of the nucleic acid sequences, or part thereof, presented in any one of FIG. 9-16.

6. An RNAi molecule according to claim 5 wherein said RNAi molecules comprise modified ribonucleotide bases.

7. (canceled)

8. A nucleic acid molecule comprising an expression cassette which cassette comprises a nucleic acid sequence which encodes at least part of a gene the sequence for which is presented in any of one FIGS. 9-16 wherein said cassette is adapted by the provision of at least one promoter such that both sense and antisense nucleic acid molecules are transcribed from said cassette.

9. A nucleic acid molecule according to claim 8 wherein said cassette is provided with at least two promoters adapted to transcribe sense and antisense strands of said nucleic acid molecule.

10. A nucleic acid according to claim 8 wherein said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

11. A nucleic acid according to claim 10 wherein said first and second parts are linked by a linker having at least one nucleotide base.

12. A nucleic acid according to claim 10 wherein said linker is at least 10 nucleotide bases.

13. (canceled)

14. A vector comprising a cassette according to claim 8.

15. A nucleic acid according to claim 8 wherein said promoter is a cancer specific promoter.

16. A nucleic acid according to claim 8 wherein said promoter is an embryonic stem cell, a germ cell or an embryonal carcinoma cell specific promoter.

17. A method to treat a condition which would benefit from an inhibition of cell division comprising administering an RNAi molecule according to Claim 5.

18. A method according to claim 17 wherein said condition is cancer.

19. A method according to claim 18 wherein said cancer is a germ cell cancer.

20. A method according to claim 19 wherein said cancer is a teratocarcinoma.

21. A method according to claim 20 wherein said cancer is selected from the group consisting of: seminoma, embryonal carcinoma, yolk sac carcinoma and choriocarcinoma.

22. A pharmaceutical composition, comprising an RNAi molecule, nucleic acid molecule or vector according to claim 5 and a carrier or excipient.

23. The method according to claim 22A, wherein said cancer is a germ cell cancer.

24. The method according to claim 23 wherein said cancer is a teratocarcinoma.

25. The method according to claim 24 wherein said teratocarcinoma is selected from the group consisting of: seminoma, embryonal carcinoma, yolk sac carcinoma and choriocarcinoma.

26. A method of treating cancer or inhibiting cell division in a subject, comprising contacting a cell of the subject with the pharmaceutical composition of claim 22.


SEQUENCE LISTING (31 sequences)

SEQ ID NO: 1 - DNA, 23 bp, T7 Phage
taatacgactcactatagggaga

SEQ ID NO: 2 - DNA, 21 bp, homo sapiens
cguaaacggccacaaguuctt

SEQ ID NO: 3 - DNA, 21 bp, Homo sapiens
gaacuuguggccguuuacgtt

SEQ ID NO: 4 - DNA, 21 bp, Homo sapiens
gauucagguuuacucacgutt

SEQ ID NO: 5 - DNA, 23 bp, Homo sapiens
acgugaguaaaccugaaucdtdt

SEQ ID NO: 6 - DNA, 21 bp, Homo sapiens
agcagcuugggcucgagaatt

SEQ ID NO: 7 - DNA, 21 bp, Homo sapiens
uucucgagcccaagcugcutt

SEQ ID NO: 8 - DNA, 22 bp, Homo sapiens
atgggcgggacatgggcatcca

SEQ ID NO: 9 - DNA, 20 bp, Homo sapiens
ggccccgggagtcgggatgg

SEQ ID NO: 10 - DNA, 20 bp, Homo sapiens
cctccgctgggcttcattcc

SEQ ID NO: 11 - DNA, 23 bp, Homo sapiens
tgggggttctgcagtctttggtc

SEQ ID NO: 12 - DNA, 32 bp, Homo sapiens
atctggcaccacaccttctacaatgagctgcg

SEQ ID NO: 13 - DNA, 32 bp, Homo sapiens
cgtcatactcctgcttgctgatccacatctgc

SEQ ID NO: 14 - DNA, 1158 bp, Homo sapiens
gtagtcctttgttacatgcatgagtcagtgaacagggaatgggtgaatgacatttgtgggtaggttatttctagaagttaggtgggcagctcggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtgccccaggctatgggagccctcacttcactgcactgtactcctcggtccctttccctgagggggaagcctttccccctgtctctgtcaccactctgggctctcccttgcattcaaactgaggtgcctgcctgcccttctaggaatgggggacagggggaggggaggagctagggaaagaaaacctggagtttgtgccagggtttttggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcttgggacacagtagataga

SEQ ID NO: 15 - DNA, 2518 bp, Homo sapiens
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 16 - DNA, 21 bp, Homo sapiens
caacuccaugaccagcucgtt

SEQ ID NO: 17 - DNA, 21 bp, Homo sapiens
cgagcuggucauggaguugtt

SEQ ID NO: 18 - DNA, 1219 bp, Homo sapiens
gggagcgggcgagtaggagggggcgccgggctatatatatagcggcctcggcctcgggcgggcctggcgctcagggaggcgcgcactgctcctcagagtcccagctccagccgcgcgctttccgcccggctcgccgctccatgcagccggggtagagcccggcgcccgggggccccgtcgcttgcctcccgcacctcctcggttgcgcactcccgcccgaggtcggccgtgcgctcccgcgggacgccacaggcgcagctctgccccccagcttcccgggcgcactgaccgcctgaccgacgcacgccctcgggccgggatgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtgaccctccagaggacccttgcctcagcctcgggaagcccctgggagggcagtgcgagggtcaccttggtgcactttcttcggatgaagagtttaatgcaagagtaggtgtaagatatttaaattaattatttaaatgtgtatatattgccaccaaattatttatagttctgcgggtgtgttttttaattttctggggggaaaaaaagacaaaacaaaaaaccaactctgacttttctggtgcaacagtggagaatcttaccattggatttctttaacttgt

SEQ ID NO: 19 - DNA, 3430 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaaaaaaaaaaa

SEQ ID NO: 20 - DNA, 2114 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcctatccagtcaatctcatggagggtggagtatggttggagcctaatcagcgaggtttctttttttttttttcctattggatcttcctggagaaaatacttttttttttttttttgagacggagtcttgctctgtcgcccaggctggagtgcagtggcgcggtcttggctcactgcaagctccgcctcccgggttcacgccattctcctgcctcagcctcccgagcagctgggactacaggcgcccgccacctcgcccggctaatattttgtatttttagtagagacagggtttcactgtgttagccaggatggtctcgatctcctgaccttgtgatccgcccgcctcggcctccctaacagctgggattacaggcgtgagccaccgcgccctgcctagaaaagacattttaataaccttggctgctaaggacaacattgatagaagccgtctctggctatagataagtagatctaatactagtttggatatctttagggtttagaatctaacctcaagaataagaaatacaagtacgaattggtgatgaagatgtattcgtattgtttgggattgggaggctttgcttatttttttaaaactattgaggtaaagggttaagctgtaacatacttaattgatttcttaccgtttttggctctgttttgctatatcccctaatttgttggttgtgctaatctttgtagaaagaggtcttgtatttgctgcatcgtaatgacatgagtactactttagttggtttaagttcaaatgaatgaaacaaatatttttcctttagttgattttaccctgatttcaccgagtgtttcgatgagtaaatatacagcttaaacat

SEQ ID NO: 21 - DNA, 2033 bp, Homo sapiens
ggagaatccccggaaaggctgagtctccagctcaaggtcaaaacgtccaaggccgaaagccctccagtttcccctggacgccttgctcctgcttctgctacgaccttctggggaaaacgaatttctcattttcttcttaaattgccattttcgctttaggagatgaatgttttcctttggctgttttggcaatgactctgaattaaagcgatgctaacgcctcttttccccctaattgttaaaagctatggactgcaggaagatggcccgcttctcttacagtgtgatttggatcatggccatttctaaagtctttgaactgggattagttgccgggctgggccatcaggaatttgctcgtccatctcggggatacctggccttcagagatgacagcatttggccccaggaggagcctgcaattcggcctcggtcttcccagcgtgtgccgcccatggggatacagcacagtaaggagctaaacagaacctgctgcctgaatgggggaacctgcatgctggggtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagttggcatctgcctttctatacaaagctactattaatcgacattgacctatttccagaaatacaattttagatatcatgcaaatttcatgaccagtaaaggctgctgctacaatgtcctaactgaaagatgatcatttgtagttgccttaaaataatgaatacaatttccaaaatggtctctaacatttccttacagaactacttcttacttctttgccctgccctctcccaaaaaactacttcttttttcaaaagaaagtcagccatatctccattgtgcctaagtccagtgtttctttttttttttttttttgagacggagtctcactctgtcacccaggctggactgcaatgacgcgatcttggttcactgcaacctccgcatccggggttcaagccattctcctgcctaagcctcccaagtaactgggattacaggcatgtgtcaccatgcccagctaatttttttgtattttagtagagatgggggtttcaccatattggccagtctggtctcgaactctgaccttgtgatccatcgatcagcctctcgagtgctgagattacacacgtgagcaactgtgcaaggcctggtgtttcttgatacatgtaattctaccaaggtcttcttaatatgttcttttaaatgattgaattatatgttcagattattggagactaattctaatgtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccatttttaaaatacattgtatatatgtgtatgcacagtaaaaatggaaactatattgacctaaaaaaaaaaaaa

SEQ ID NO: 22 - DNA, 1224 bp, Homo sapiens
ggagctctccccggtctgacagccactccagaggccatgcttcgtttcttgccagatttggctttcagcttcctgttaattctggctttgggccaggcagtccaatttcaagaatatgtctttctccaatttctgggcttagataaggcgccttcaccccagaagttccaacctgtgccttatatcttgaagaaaattttccaggatcgcgaggcagcagcgaccactggggtctcccgagacttatgctacgtaaaggagctgggcgtccgcgggaatgtacttcgctttctcccagaccaaggtttctttctttacccaaagaaaatttcccaagcttcctcctgcctgcagaagctcctctactttaacctgtctgccatcaaagaaagggaacagttgacattggcccagctgggcctggacttggggcccaattcttactataacctgggaccagagctggaactggctctgttcctggttcaggagcctcatgtgtggggccagaccacccctaagccaggtaaaatgtttgtgttgcggtcagtcccatggccacaaggtgctgttcacttcaacctgctggatgtagctaaggattggaatgacaacccccggaaaaatttcgggttattcctggagatactggtcaaagaagatagagactcaggggtgaattttcagcctgaagacacctgtgccagactaagatgctcccttcatgcttccctgctggtggtgactctcaaccctgatcagtgccacccttctcggaaaaggagagcagccatccctgtccccaagctttcttgtaagaacctctgccaccgtcaccagctattcattaacttccgggacctgggttggcacaagtggatcattgcccccaaggggttcatggcaaattactgccatggagagtgtcccttctcactgaccatctctctcaacagctccaattatgctttcatgcaagccctgatgcatgccgttgacccagagatcccccaggctgtgtgtatccccaccaagctgtctcccatttccatgctctaccaggacaataatgacaatgtcattctacgacattatgaagacatggtagtcgatgaatgtgggtgtgggtaggatgtcagaaatgggaatagaaggagtgttcttagggtaaatcttttaataaaactacctatctggtttatgaccacttagatcgaaatgtca

SEQ ID NO: 23 - DNA, 3494 bp, Homo sapiens
ggcacccttcggcgagcgctgtttgtttagggctcggtgagtccaatcaggagcccaggctgcagttttccggcagagcagtaagaggcgcctcctctctcctttttattcaccagcagcgcggcgcagaccccggactcgcgctcgcccgctggcgccctcggcttctctccgcgcctgggagcaccctccgccgcggccgttctccatgcgcagcgcccgcccgaggagctagacgtcagcttggagcggcgccggaccgtggatggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctgagcccacgccgccaggaggcagggagggctccgccgcgggcctcactccactcgtgtctgcttttgtgcagcggtccagacagtggcgactgcgctgacagaacgtgattctcgtgcctttattttgaaagagatgtttttcccaagaggcttgctgaaagagtgagagaagatggaagggaagggccagtgcaactgggcgcttgggccactccagccagcccgcctccggggcggaccctgctccacttccagaagccaggactaggacctgggccttgcctgctatggaatattgagagagattttttaaaaaagattttgcattttgtccaaaatcatgtgcttcttctgatcaattttggttgttccagaatttcttcataccttttccacatccagatttcatgtgcgttcatggagaagatcacttgaggccatttggtacacatctctggaggctgagtcggttcatgaggtctcttatcaaaaatattactcagtttgcaagactgcattgtaactttaacatacactgtgactgacgtttctcaaagttcatattgtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaatttttttttttgaatgggatgtcctatggaaacctatttcaccagagttttaaaaataaaaagggtattgttttgtcttctgtacagtgagttccttcccttttcaaagctttctttttatgctgtatgtgactatagatattcatataaaacaagtgcacgtgaagtttgcaaaatgctttaaggccttcctttcaaagcatagtccttttggagccgttttgtaccttttataccttggcttatttgaagttgacacatggggttagttactactctccatgtgcattggggacagtttttataagtgggaaggactcagtattattatatttgagatgataagcattttgtttgggaacaatg

SEQ ID NO: 24 - DNA, 925 bp, Homo sapiens
ggcacgagccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttg

SEQ ID NO: 25 - DNA, 1098 bp, Homo sapiens
atggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtccctcacagcttgtaaagtgtga

SEQ ID NO: 26 - DNA, 1310 bp, Homo sapiens
agacgccgagatgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctactacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgacccctgttcgcatgctgacctgtgtttcctcccca

SEQ ID NO: 27 - DNA, 1549 bp, Homo sapiens
gaattcgggggggagatgcgggtcatggcgccccgaaccctcatcctgctgctctcgggagccctggccctgaccgagacctgggccggctcccactccatgaggtatttctccacatccgtgtcctggcccggccgcggggagccccgcttcatcgcagtgggctacgtggacgacacgcagttcgtgcggttcgacagcgacgccgcgagtccaagaggggagccgcgggagccgtgggtggagcaggaggggccggagtattgggaccgggagacacagaagtacaagcgccaggcacaggctgaccgagtgaacctgcggaaactgcgcggctactacaaccagagcgaggacgggtctcacaccctccagaggatgtttggctgcgacctggggccggacgggcgcctcctccgcgggtataaccagttcgcctacgacggcaaggattacatcgccctgaacgaggatctgcgctcctggaccgccgcggacacggcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtggtggtgccttctggagaagagcagagatacacgtgccatgttcagcacgaggggctgccggagcccctcaccctgagatggaagccgtcttcccagcccaccatccccatcgtgggcatcgttgctggcctggctgtcctggctgtcctagctgtcctaggagctatggtggctgttgtgatgtgtaggaggaagagctcaggtggaaaaggagggagctgctctcaggctgcgtccagcaacagtgcccagggctctgatgagtctctcatcgcttgtaaagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggagagacagcccacccccgtgtccaccgtgacccctgtccccacactgacctgtgttccctccccgatcatctttcctgttccagagaagtgggctggatgtctccatctctgtctcaacttcatggtgcgctgagctgcaacttcttacttccctaatgaagttaagaacctgaatataaatttgttttctcaaatatttgctatgaagggttgatggattaattaaataagtcaattcctggaagttgagagagcaaataaagacctgagaaccttccaaaaacccgcccgaattc

SEQ ID NO: 28 - DNA, 1095 bp, Homo sapiens
atggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggacattttcttccaacaggtggaaaaggagggagctactctaaggctgagtggagcgacagtgcccaggggtctgagtctcacagcttgtaa

SEQ ID NO: 29 - DNA, 1188 bp, Homo sapiens
atggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtcactgacagtgcccagggctctggggtgtctctcacagctaataaagtgtgagacagcttccttgtgtgggactgagaagcaagatatcaatgtagcagaattgcacttgtgcctcacgaacatacataaattttaaaaataaagaataaa

SEQ ID NO: 30 - DNA, 1840 bp, Homo sapiens
cccattaggtgacaggtttttagagaagccaatcacgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaagtaagtatgaaggaggctgatccctgagatccttgggatcttgtgtttgggagccatgggggagctcacccaccccacaattcctcctctggccacatctcctgtggtctctgaccaggtgctgtttttgttctactctaggcagtgacagtgcccagggctctaatgtgtctctcacggcttgtaaatgtgacaccccggggggcctgatgtgtgtgggttgttgaggggaacaggggacatagctgtgctatgaggtttctttgacttcaatgtattgagcatgtgatgggctgtttaaagtgtcacccctcactgtgactgatatgaatttgttcatgaatatttttctgtagtgtgaaacagctgccctgtgtgggactgagtggcaagtccctttgtgacttcaagaaccctgacttctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaa

SEQ ID NO: 31 - DNA, 7201 bp, Homo sapiens
atgaccgctttggaaaaacaaagactgtatttcctggaaattaatgtttattcaataaactgtgtattcagctatatcacatagtggtgaggctgaaatgaggcgggaagaggcggttggggcttaattatatcaatttgggtggccccacagcgcctccaaggcgccagtcctgttttgacaagttgcctctggaagcctctacaatgcctctcttctttttctccagagtaagcggaggccaggggcccccggcctctgcttaatactaaaaaaaacagctgttgtcatagtaatgattgggtggaaacattccaggcctgggtggagaggctttttgcttcctcttgcaaaaccacactgacattccaggcctgggtggagaggctttttgcttcctcttgcaaaaccacactgccctctggagggcagttgcctagcaactaactaaaagaggatgtcgcacggccagctgcggtcagttagtcacttcctgcttaactgacttgacattttctattttaagagtcgggaggaaaattactgtgttggaggccctccgccatcttctgaagctgaatcgaattaacttgtttattgcagcttataatggttacaaataaagcaatagcatcacaaatttcacaaataaagcatttttttcactgcattctagttgtggtttgtccaaactcatcaatgtatcttatcatgtctggatctgatatcatcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctcgataagcttgatatcgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttca